(19) United States
(12) Patent Application Publication
(10) Pub. No.: US 2010/0312119 A1
(43) Pub. Date: Dec. 9, 2010

(54) ULTRASONIC PROBE AND ULTRASONIC IMAGING APPARATUS

(76) Inventors: Kunio Hashiba, Tokyo (JP); Mikio Izumi, Soka (JP); Katsunori Asafusa, Kashiwa (JP)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 12/744,996

(22) PCT Filed: Nov. 25, 2008
(86) PCT No.: PCT/JP2008/003454

§ 371 Date: May 27, 2010

(30) Foreign Application Priority Data
Nov. 28, 2007 (JP) 2007-306747

(51) Int. Cl. A61B 8/14

(52) U.S. Cl. 600/459

(57) ABSTRACT

There is provided an ultrasound probe including a capacitive transducer for reducing multiple reflections while maintaining a pulse characteristic with which a high-quality image can be obtained, and is also provided an ultrasound imaging apparatus using the ultrasound probe. The ultrasound probe including the capacitive transducer is configured so as to satisfy the condition 6.5/f_{c}<αd, where α [dB/mm/MHz] denotes the absorption coefficient of an acoustic lens, d [mm] denotes the maximum thickness of the acoustic lens, and f_{c} [MHz] denotes the center frequency of the capacitive transducer, and satisfy the condition L<1/((3πf_{c})²×C), where L [H] denotes an inductance value per channel of the capacitive transducer, C [pF] denotes a capacitance per channel of the capacitive transducer, and f_{c} [MHz] denotes the center frequency of the capacitive transducer.

### TECHNICAL FIELD

The present invention relates to an ultrasound probe including a capacitive transducer so as to obtain a high-quality image, and an ultrasound imaging apparatus employing the capacitive transducer.

### BACKGROUND ART

A capacitive transducer has been proposed as a transducer array that is more highly efficient and more broadband than a conventional piezoelectric transducer.

The capacitive transducer is, as described in, for example, non-patent document 1, structured to have numerous diaphragms formed on a silicon substrate. The capacitive transducer is used with a DC bias voltage applied to electrodes formed on each of the diaphragms and silicon substrate. With the voltage applied, an AC voltage is further applied in order to transmit sound waves. Arriving sound waves are received with the DC bias voltage applied.

When the DC bias voltage is stepped up, the diaphragms are brought into contact with the silicon substrate due to Coulomb force. The DC bias voltage at this time is called a collapse voltage. The DC bias voltage that is equal to or lower than the collapse voltage is normally applied for use.

In the capacitive transducer, as described in, for example, non-patent document 2, the conversion efficiency between electric energy and mechanical energy (electromechanical coupling factor) is known to depend on the DC bias voltage.

Non-patent document 1: “A Surface Micromechined Electrostatic Ultrasonic Air Transducer” (Proceedings of 1994 IEEE Ultrasonics Symposium, pp. 1241-1244)

Non-patent document 2: “A Single Distributed Model for cMUT” (Proceedings of 2004 IEEE Ultrasonics Symposium, pp. 248-251)

### DISCLOSURE OF THE INVENTION

### Problems to be Solved by the Invention

The electrostatic transducer is a capacitive transducer, and a large parasitic capacitance is present in the electrostatic transducer. The DC bias voltage equal to or lower than the collapse voltage causes the electromechanical coupling factor to be so small as to hinder matching between an acoustic load and a receiver circuit. Part of received echoes is reflected from the transducer. The reflected waves are further reflected from a target in the acoustic load, and become receiving echoes. In case of imaging, there is a fear that image quality may be degraded by an artifact due to multiple echoes.

Even when an acoustic matching layer is formed in order to match an acoustic load with a transducer, since the matching is attained at a specific frequency determined with the material (density and speed of sound) of the acoustic matching layer and the thickness thereof, a receiving signal is a narrow-band one. In addition, a pulse exhibits a ringing because phase linearity is disturbed. Therefore, there is a fear that a pulse characteristic for providing a high resolution may be deteriorated.

In the capacitive transducer, how to cope with degradation in image quality or deterioration in a pulse characteristic is left as an unsolved problem. As mentioned above, an ultrasound probe including the capacitive transducer and an ultrasound imaging apparatus employing the ultrasound probe are confronted with an issue that an artifact caused by multiple echoes cannot be reduced while a pulse characteristic for providing a high resolution is sustained.

### Means of Solving the Problems

An example of an ultrasound probe in accordance with the present invention includes a capacitive transducer having at least one channel formed on a substrate, an acoustic lens disposed on one surface of the capacitive transducer, and a series inductor connected in series with each channel of the capacitive transducer. The acoustic lens is formed with the absorption coefficient of the acoustic lens and the maximum thickness of the acoustic lens based on the resonant frequency of the capacitive transducer. As for the inductance value of the series inductor, the inductance value per channel of the capacitive transducer is specified based on the electrostatic capacity per channel of the capacitive transducer and the center frequency of the capacitive transducer.

An example of an ultrasound imaging apparatus in accordance with the present invention includes: an ultrasound probe that includes a capacitive transducer having plural channels formed on a substrate, an acoustic lens disposed on one surface of the capacitive transducer, and an inductor connected to the capacitive transducer; a DC electrical source that applies a DC bias to the ultrasound probe; an AC electrical source that applies an AC voltage; and a receiver circuit that processes a receiving signal of the ultrasound probe.

### EFFECTS OF THE INVENTION

According to the invention, an ultrasound probe including a capacitive transducer capable of reducing an artifact due to multiple echoes while sustaining a pulse characteristic for providing a high resolution, and an ultrasound imaging apparatus employing the ultrasound probe can be provided.

### BEST MODE FOR CARRYING OUT THE INVENTION

Referring to the drawings, an embodiment of an ultrasound probe in accordance with the present invention will be described below.

FIG. 1 is a configuration diagram showing an embodiment of an ultrasound probe in accordance with the present invention. The ultrasound probe of the present embodiment includes plural capacitive transducer array channels 100 (100A, 100B, 100C, etc.). One inductor 21A, 21B, 21C, or the like is connected in series with each array channel.

The mechanical component of each channel, for example, the channel 100A includes plural diaphragm structures 10A, 10B, 100, etc. formed on a silicon substrate 11 through a silicon process, and an acoustic lens 17 disposed on one surface of the capacitive transducer 10.

One diaphragm structure 10A (or 10B or 10C) included in the capacitive transducer 10 has a diaphragm 12 formed by way of ribs 16. A layer of cavity 15 is formed between the diaphragm 12 and silicon substrate 11.

An upper electrode 14 is contained in the diaphragm 12, and a lower electrode 13 is contained in the silicon substrate 11. A DC bias voltage is applied to the lower electrode 13 by a DC electrical source 20. For use, the diaphragm 12 is deformed toward the silicon substrate due to Coulomb force. The DC bias voltage is normally set to a voltage value that ranges from about 80% to 90% of a voltage (the collapse voltage) bringing the diaphragm 12 into contact with the silicon substrate 11.

In general, transducers included in an ultrasound probe for acquiring a two-dimensional ultrasound image have a one-dimensional array structure composed of plural channels arranged like strips. The ultrasound probe of the present embodiment has the array structure of plural channels. One channel has plural diaphragm structures 10A, 10B, 10C, etc. concatenated in parallel. Specifically, the upper electrodes 14 included in the diaphragms 12 of the respective diaphragm structures 10A, 10B, 10C, etc. are connected in parallel with one another within the channel. One inductor 21 is connected in series with one channel. In the present embodiment, since a DC voltage is applied to the lower electrode, the inductor 21 is connected to the upper electrodes 14. The terminal of the series inductor 21 opposite to the terminal thereof on the side of the capacitive transducer is connected to transmitter/receiver circuits (T/R circuits) 23 of a control circuit 26, each of which is included in an ultrasound imaging apparatus, over a probe cable 22 having a length of about 2 m. If the ultrasound probe is for medical use, it usually includes plural channels, for example, includes an array of one hundred channels, and one hundred inductors. Therefore, the probe cable in FIG. 1 has plural lines bundled therein, and the transmitter/receiver circuits 23 includes the same number of transmitter receivers as the number of array channels. For brevity's sake, the transmitter/receiver circuit is shown as one transmitter/receiver circuit. For transmitting ultrasound waves, the transmitter/receiver circuit 23 operates so that an AC electrical source 24 for transmission can be connected to the series inductors 21. For receiving ultrasound waves, the transmitter/receiver circuit 23 operates so that a receiver circuit 25 can be connected to the series inductors 21. For the operations, a diode switch or the like may be included, or a multiplexer may be used to allow a controller 27 to control a connected state.

For some usages including a usage for non-destructive testing, a one-channel ultrasound probe may be used to perform a mechanical scan. In this case, one series inductor 21 is connected to one capacitive transducer array channel 100 included in the ultrasound probe.

Next, an electrical equivalent circuit of the capacitive transducer shown in FIG. 1 is used to describe a reverberation occurring during reception. FIG. 2 is a diagram of an electrical equivalent circuit for reception equivalent to one channel of the capacitive transducer 10 shown in FIG. 1. The capacitive transducer 10 is excited by driving force 41 (F) by arriving sound as receiving echoes, with an acoustic load impedance 40 (z_{w}) of a living body or the like as a resistance.

An electrical equivalent circuit 30 of the capacitive transducer 10 has a mechanical impedance 32 (zₘ) of the diaphragms 12 and a capacitance C of a capacitor 31 electromechanically converted with a force factor φ. The force factor φ is a factor whose dimension is [N/V] and related to an electromechanical coupling factor. The larger the force factor value is, the higher the conversion efficiency between electric energy and mechanical energy is. The electromechanical coupling factor is electrically written as a transformer 33 of a winding ratio φ:1. Therefore, the acoustic load impedance 40 and the mechanical impedance 32 of the diaphragms 12 are written as electrical impedances z_{w}/φ² and zₘ/φ² respectively. A receive impedance 42 is connected to a terminal on the side of the capacitor 31, and received as an electric signal. A series inductor 43 (inductance value L) that is a feature of the present invention is inserted to a terminal B in FIG. 2, that is, between the capacitor 31 and the receive impedance 42. Normally, when the electric impedance of the probe observed at a BB′ end (on the left side of the BB′ end in FIG. 2) is squared with the receive impedance 42, the energy transmission efficiency is maximized. In the present invention, the series inductor 43 is used to perform tuning so that the energy transmission efficiency can be upgraded without impairment of a feature that the phase linearity of the capacitive transducer is excellent over a broad frequency band.

In FIG. 2, an issue of a conventional ultrasound probe that does not have a series inductor connected (electrical matching is not attained) will be described below. The acoustic load impedance 40 per one channel is expressed approximately as z_{w}=r_{w}+jωI_{w} in relation to a forward resistance and an inductance. Herein, ω denotes a frequency, and j denotes an imaginary unit. An inductance component I_{w} is derived from the fact that since the width of one channel of the capacitive transducer 10 included in the array is about ½ of a wavelength at an employed frequency, a load of a plane wave is not identified. In contrast, the nature of the diaphragms 12 as a mechanical vibrating membrane for one channel can be represented by the mechanical impedance 32 (zₘ) or represented by a series circuit of a forward resistance rₘ representing a mechanical loss, an inductance Iₘ representing a mass effect of the diaphragms 12, and a capacitor cₘ representing a compliance of the diaphragms 12, and expressed as an equation (1).

Zₘ=rₘ+j(ωIₘ−1/ωcₘ) (1)

ω under ωIₘ−1/ωcₘ=0 denotes a mechanical resonant angular frequency of the diaphragms 12.

Next, a scattering parameter (S-parameter) S will be described below in order to discuss to what extent energy induced by the driving force 41 by arriving sound is reflected from the capacitive transducer. In the electrical equivalent circuit diagram of FIG. 2, reflection from an AA′ end of the part of electrical equivalent circuit 30 should merely be discussed. Assuming that a wave incident on the electrical equivalent circuit 30 of the capacitive transducer from the side of the acoustic load impedance 40 is “a” and a reflected wave is “b”, the scattering parameter S is expressed as an equation (2) by regarding an impedance of the entire electrical equivalent circuit 30 of the capacitive transducer, which is observed at the AA′ end, as Zᵢₙ.

S=b/a=(Zᵢₙ−Z_{w}*)/(Zᵢₙ+Z_{w}) (2)

Herein, under Z_{w}=z_{w}/φ², the acoustic load impedance zw is expressed as an electric impedance. In addition, * denotes a conjugate complex number.

A capacitive transducer in which the mechanical resonant frequency of the diaphragms 12 is approximately 8 MHz is reviewed through calculation based on a finite element method. For a conventional type of ultrasound probe in which a series inductor is not connected, constants of the equivalent circuit shown in FIG. 2 are calculated. As a result, the electrical equivalent circuit constants shown in FIG. 3 are obtained. FIG. 4 is a characteristic diagram showing the scattering parameter S in relation to a frequency on the basis of the values in FIG. 3. FIG. 4 is based on the assumption that the receive impedance Rᵢₙ is equal to a real number of the acoustic load impedance Z_{w}.

As apparent from FIG. 4, when the capacitance C equals 66 pF, the value of the scattering parameter S is very large. A reflected wave of about 90% (when the incident wave is 0 dB, the reflected wave is approximately −1 dB) is present even at a frequency near the resonant frequency. This is because the impedance of the capacitance C of the capacitor 31 is large for the acoustic load impedance. In FIG. 4, results obtained when the capacitance C gets smaller than 66 pF are shown on a simulated basis. Unless the capacitance C is considerably small, no effect is exerted. Even when the capacitance C is 0, a reactance component of the mechanical impedance zₘ of the diaphragms 12 cannot be canceled. An effect is seen to be confined to a narrow band.

In contrast, in order to attain acoustic matching between an acoustic load impedance and a transducer, an acoustic matching layer is generally employed in a probe using a PZT. However, although an effect is exerted at a specific frequency determined with the material and thickness of the matching layer, the employment of the matching layer becomes a cause of disturbing the phase linearity at a frequency near the specific frequency in a wide frequency band, and deteriorates the pulse characteristic. Namely, the pulse characteristic is degraded to the one that a ringing is present, and often causes a blur in an image.

As mentioned above, in the ultrasound probe including the capacitive transducer, intense multiple echoes occur due to the low electroacoustic conversion efficiency. It is hard to reduce the multiple echoes while sustaining the pulse characteristic that a ringing is absent in a wide frequency band and that is specific to the capacitive transducer.

In consideration of the foregoing problems, an ultrasound probe of an embodiment of the present invention has reduction of multiple echoes by an acoustic lens and improvement of sensitivity through insertion of a series inductor combined under a specific condition for the purpose of minimizing multiple echoes while sustaining the pulse characteristic of a little ringing that is a feature of the capacitive transducer.

FIG. 5 is a schematic diagram showing reduction of multiple echoes by an acoustic lens to which a series inductor is not connected. Sound waves radiated from the capacitive transducer 10 are passed through the acoustic lens 17 and propagated to an acoustic load such as a living body that is not shown. A first reflection echo 51 reflected from a certain target in the acoustic load is passed through the acoustic lens 17, and received by the capacitive transducer 10. As described in conjunction with FIG. 4, a portion of the first reflection echo to be converted into electric energy as a first receive echo 52 by the capacitive transducer is about 10% in spite of the center frequency thereof. More particularly, since an amplitude ratio of pulses has a significant meaning, the amplitude ratio of the first receive echo 52 to the first reflection echo 51 is calculated on the assumption that the first reflection echo 51 whose center frequency is 8 MHz, which numbers two waves, and which is Hanning-weighted has arrived at the capacitive transducer 10 exhibiting the electrical equivalent circuit constants shown in FIG. 3. This results in:

(Amplitude of the first receive echo 52)/(Amplitude of the first reflection echo 51)=0.55

Assuming that the amplitude of the first reflection echo is 0 dB, a pulse component having an amplitude of −7 dB is reflected, passed through the acoustic lens 17, and radiated again to the acoustic load that is not shown. The re-radiated pulse is reflected from a certain target in the acoustic load. The multiple reflection echo 53 is passed through the acoustic lens 17, and received as a receive echo 54 by multiple reflection, which has an amplitude that is about 0.55 times larger than the amplitude of the multiple reflection echo 53, by the capacitive transducer.

Mathematically, assuming that the one-way attenuation factors at the acoustic lens 17 and the acoustic load that is not shown are α′ and β′ respectively, the ratio of the amplitude of the receive echo 54 by multiple reflection to that of the first receive echo 52 is expressed as an equation (3).

(Amplitude of the receive echo 54 by multiple reflection)/(Amplitude of the first receive echo 52)=0.45α′²β′² (3)

In general, the absorption coefficient of a living body ranges from 0.7 to 1 dB/cm/MHz. In a frequency band usually employed in ultrasound imaging (from about 1 to 15 MHz), when a perfect reflecting body lies at a distance of 1 cm, up to about 30 dB is attenuated. If a ringing of a pulse is larger by about −20 dB than the size of a main pulse, a blur in an image often becomes outstanding. Therefore, if attenuation of about −20 dB is attained only through attenuation by the acoustic lens 17, an artifact due to multiple echoes is thought to be suppressed to such an extent that it will not affect image quality. Namely, an inequality (4) should be established.

20 Log₁₀(0.45α′²)<−20 (4)

Therefore, when the absorption coefficient of the acoustic lens 17 is α [dB/mm/MHz] and the maximum lens thickness is d [mm], assuming that the center frequency of the capacitive transducer is f_{c} [MHz], a converted value in dB of the on-way attenuation factor α′ at the acoustic lens 17, 20 Log₁₀(α′), is squared with −αdf_{c}. Herein, the conditional inequality (4) for suppressing an artifact due to multiple echoes is deformed into an inequality (5) below.

20 Log₁₀(0.45)+2×20 Log₁₀(α′)<−20 (5)

Then, 20 Log₁₀ (α′)=−αdf_{c} is assigned to the inequality (5), whereby an inequality (6) is obtained.

20 Log₁₀(0.45)−2αdf_{c}<−20 (6)

The inequality is straightened in order to obtain an inequality (7) below.

6.5/f_{c}<αd (7)

Therefore, when the inequality (7) is satisfied, multiple echoes can be suppressed by the acoustic lens 17.

The foregoing reduction of multiple echoes depending on the thickness of the acoustic lens is attributable to the fact that the number of times by which the acoustic lens is passed through along a sound-wave propagation path is different between the receive echo 54 by multiple reflection and first receive echo 52. By thickening the acoustic lens 17, the first receiving echo necessary to perform imaging is affected by attenuation. This poses an issue of a decrease in sensitivity.

In the ultrasound probe of the present embodiment, as shown in the equivalent circuit in FIG. 2, the series inductor 43 (inductance value L) is inserted in order to cancel the capacitance C of the capacitor 31 so as to improve sensitivity. However, as described in conjunction with FIG. 4, even if the capacitance C is fully canceled, a broadband signal is not obtained. In the ultrasound probe of the present embodiment, matching with the fractional bandwidth of the capacitive transducer is attained at a frequency near an upper-limit frequency of a high frequency by means of the series inductor 21.

Herein, the impedance Z of a series circuit including a resistor R, an inductor L, and a capacitor C is written as an equation (8).

Z=R+jωL+(1/jωC)=R+j(ωL−(1/ωC)) (8)

where ω (=2πf) denotes an angular frequency.

According to the circuit constants employed in FIG. 3, the impedance is very high for a sensor, and it is hard to attain electrical matching with a receiver circuit. In order to improve sensitivity, the absolute value of Z is thought to be decreased. In order to minimize the absolute value of Z, the imaginary number should be made null. The condition is expressed as ω=1/√(LC), that is, a condition for series resonance. Thus, by inserting the series inductor 43, the capacitance of the capacitor C can be canceled. In this case, C denotes the capacitance of the impedance on the side of the probe observed at the BB′ end. In practice, the influence of the capacitor 31 is significant. When the capacitance of the capacitor 31 is large, the impedance of the capacitor 31 diminishes. A current largely flows into the capacitor, and the receiver circuit does not exhibit receiving sensitivity. In this case, even cancelation of the capacitor 31 is effective. A decrease in the impedance Z attained by inserting the series inductor 43 is maximized under the condition for series resonance. A remedial effect is markedly manifested at a series resonant frequency, but is hardly exerted at the other frequencies. Therefore, if the series resonant frequency falls within the frequency band for the sensor, the frequency band is narrowed. Therefore, in the present invention, as described below, the angular frequency ω is varied (shifted from the resonant frequency) in order to exert the remedial effect for receiving sensitivity over a wide frequency band.

The frequency band of the capacitive transducer is wide, and a capacitive transducer exhibiting a fractional bandwidth of about 100% can be manufactured. The broadband frequency characteristic is accompanied by the advantages that not only an amplitude characteristic but also phase linearity are excellent, that is, a group delay characteristic is flat. In FIG. 1, assuming that the center frequency of the capacitive transducer 10 is f_{c}, when the fractional bandwidth is 100%, the upper-limit frequency of the bandwidth is 1.5f_{c}. Assuming that the inductance value of the series inductor 21 is L, a condition for canceling the capacitance C using the series inductor 21 at the frequency is expressed as an equation (9) providing the condition for series resonance of LC. The equation (9) is deformed in order to obtain an equation (10) below.

1.5f_{c}=1/(2π(LC)^{0.5}) (9)

L=1/((3πf_{c})²×C) (10)

Under the condition under which LC resonance occurs at a frequency equal to or lower than 1.5f_{c}, the LC resonance occurs within the frequency band of the capacitive transducer. There is a fear that the flat group delay characteristic may be disturbed. The LC resonant frequency should therefore be higher than 1.5f_{c}. Namely, the inductance value L of the series inductor 21 should satisfy a condition expressed by an inequality (11) below.

L<1/((3πf_{c})²×C) (11)

In the above inequality, the dimensions of the center frequency f_{c} and capacitance C are megahertz and picofarads respectively.

As mentioned above, when the capacitance C is canceled using the series inductor 21, the phase linearity is degraded at a frequency near the frequency of 1.5f_{c}. As for the amplitude characteristic, peaking is arisen to cause the pulse characteristic to indicate occurrence of a ringing. However, due to attenuation by the acoustic lens 17, a high-frequency component undergoes a high attenuation effect. Therefore, although the fractional bandwidth is a bit narrowed, while the pulse characteristic that the group delay characteristic is flat is sustained, sensitivity can be improved.

In an ultrasound probe employing a piezoelectric ceramic such as PZT, a multilayer matching layer is used to take measures for expanding a bandwidth. There is difficulty in manufacturing an ultrasound probe whose group delay characteristic is flat and whose fractional bandwidth is 100% or more.

As a material of the acoustic lens 17, for an acoustic load such as a living body, the intrinsic acoustic impedance of the acoustic lens 17 is preferably squared with that of the acoustic load such as the living body for fear reflection may occur on the border between the acoustic lens 17 and acoustic load. For example, the sound speed of silicon rubber ranges from about 900 to 1000 m/s, and is about ⅔ of the sound speed of the living body. Further, the density of silicon rubber is about 1.3 to 1.4 times larger than that of the living body. The intrinsic acoustic impedance of silicon rubber is nearly equal to that of the living body. Silicon rubber is therefore an appropriate material.

The results of experiment on the ultrasound probe of the present embodiment will be described below, and the advantages of the present invention will be described below.

FIG. 6 includes pulse characteristic diagrams and frequency characteristic diagrams concerning an ultrasound probe unrelated to the embodiment of the present invention. FIG. 7 includes pulse characteristic diagrams and frequency characteristic diagrams concerning an ultrasound probe to which the embodiment of the present invention is applied. Herein, the pulse characteristic diagram expresses absolute values obtained by performing Hilbert transformation on an obtained receiving signal, or shows an envelope of pulses.

Each of (a) and (b) in FIGS. 6 and 7 show the same characteristic in different ways of expression. The pulse characteristic in each (a) of FIGS. 6 and 7 is expressed with an impulse response of a probe, and the frequency characteristic in each (b) of FIGS. 6 and 7 has the pulse characteristic of each (a) of FIGS. 6 and 7 expressed in a frequency domain. A resonant frequency is a frequency at which the mechanical impedance 32 shown in FIG. 2 takes on a value under series resonance.

To the ultrasound probe which is shown in FIG. 6 and is unrelated to the present embodiment, an acoustic lens that is made of silicon rubber and designed to be focused on width the elevational axis direction of 7.9 mm at a distance on a sound axis of 35 mm is attached. The maximum thickness d is 0.65 mm. The absorption coefficient α of silicon rubber ranges from about 0.7 to 1 dB/mm/MHz, and the resonant frequency and center frequency f_{c} of a sole capacitive transducer is about 9 MHz. Therefore, 6.5/f_{c}>αd is established. Apparently, the ultrasound probe is constituted to fall outside the range defined as the inequality (7) in the present embodiment. As apparent from FIG. 6, when the silicon rubber acoustic lens of 0.65 mm thick is attached, the center frequency is approximately 8 MHz. Namely, when the acoustic lens 17 is attached, sound waves to be actually transmitted or received are attenuated while being passed through the lens. The higher the frequency is, the larger the attenuation is. In terms of a power spectrum of the frequency characteristic, the higher frequencies are more markedly affected. Even when the center frequency of the sole capacitive transducer is 9 MHz, the center frequency of the probe including the acoustic lens is slightly lowered. In the example of FIG. 6, the center frequency is approximately 8 MHz. If the thickness of the acoustic lens 17 is further increased, the center frequency of the probe is lowered. If the thickness thereof is decreased, the center frequency of the probe approaches the center frequency of the sole transducer. In the example of FIG. 7, since the thickness of the acoustic lens is larger than that in the example of FIG. 6, the center frequency is about 7 MHz.

FIG. 8 lists the results of measurement performed in water on a transmit/receive gain and a multiple-echo level with an aluminum block disposed at an elevational focal point. As seen from FIG. 8, in the ultrasound probe (example of FIG. 6) unrelated to the present embodiment, the amplitude level of a multiple-echo signal with respect to the amplitude of the first receiving echo is −16.3 dB. There is a high possibility that the amplitude level may cause an artifact in case of imaging.

In contrast, when the constitution of the present embodiment is applied, the absorption coefficient of silicon rubber that is a material of an acoustic lens ranges from about 0.7 to 1 dB/mm/MHz, and the center frequency of a sole capacitive transducer is about 9 MHz. Therefore, under the condition of 6.5/f_{c}<αd, the maximum thickness of the acoustic lens is equal to or larger than at least about 0.73 mm, or preferably, equal to or larger than about 1.1 mm. Herein, the word “about” shall imply the extent of a manufacture error.

By increasing the thickness of an acoustic lens, a decrease in a transmit/receive gain accompanying attenuation by the acoustic lens poses a problem. When an inductance value of a series inductor to be connected in series with a capacitive transducer is obtained according to the present embodiment, the inductance value is expressed as an inequality (12) below.

L<1/((3πf_{c})²×C)=2.1 μH (12)

In the above inequality, the dimension of the center frequency f_{c} is megahertz, the dimension of the capacitance C is picofarads, and f_{c}=9 MHz and C=66 pF are adopted.

Based on the above results, the material of the acoustic lens is set to silicon rubber, the thickness thereof is set to 1.2 mm (however, the elevational focus position is 25 mm). Generally, locally procurable inductors are available in the lineup of 1.0 μH, 1.2 μH, 1.5 μH, 1.8 μH, and 2.2 μH. Therefore, assuming that the inductance value of a series inductor connected to a capacitive transducer is smaller than 2.1 μH and is 1.8 μH at maximum, the pulse characteristic and frequency characteristic are measured according to the same method as the aforesaid one.

FIG. 7 shows the results of measurement. FIG. 8 shows the measured values of a transmit/receive gain and a multiple-echo level along with the results of measurement on the ultrasound probe (example of FIG. 6) unrelated to the present embodiment.

As seen from FIG. 8, the ultrasound probe in accordance with the present embodiment has the multiple-echo level suppressed to −20 dB or less without a decrease in the transmit/receive gain.

As seen from FIG. 7, although the fractional bandwidth is slightly narrowed, degrading in phase linearity caused by insertion of an inductance is prevented, and a flat group delay characteristic is sustained. Therefore, it is seen that an excellent pulse characteristic of a little ringing is obtained. Specifically, as apparent from the comparison of FIG. 7 with FIG. 6, since the frequency band of high frequencies can be diminished by increasing the thickness of an acoustic lens, the bandwidth is slightly narrowed. When the bandwidth is narrowed, a pulse duration is generally widened and an axial resolution is degraded. When the inductance 21 is inserted, electrical matching is attained at one frequency. Therefore, a gain increases at the frequency at which matching is attained (series resonance is attained). As a result, a narrow band ensues, and the phase linearity is lost. When the phase linearity is lost, the shape of a pulse is varied. As a result, even when an incident pulse is an impulse, a ringing occurs in a received wave. This causes the axial resolution to be degraded or causes an artifact to be generated. However, according to the present embodiment, degradation in the phase linearity caused by insertion of an inductance can be prevented, and the flat group delay characteristic can be sustained. Therefore, image quality is hardly affected.

As presented as the embodiment above, the ultrasound probe of the present invention can acquire a high-quality image with a little blur or a little artifact due to multiple echoes.

For the main receiving echo intensity to be originally used as image data, the receiving echo intensity due to multiple reflections is suppressed through attenuation by an acoustic lens. Therefore, a high-quality ultrasound image having an artifact due to multiple echoes suppressed can be obtained.

A series inductance is inserted so that matching can be attained at a frequency near an upper-limit frequency of a frequency band for a capacitive transducer included in an ultrasound probe. While a pulse characteristic of a little ringing is sustained in order to obtain image quality of a high resolution, the sensitivity for a main receiving echo can be improved through attenuation by an acoustic lens.

The condition for an inductance may be expanded from the center frequency to a bandwidth.

Assuming that the absorption coefficient of the acoustic lens is α [dB/mm/MHz], the maximum thickness of the acoustic lens is d [mm], the center frequency of the capacitive transducer is f_{c} [MHz], and reflection from an interface of the capacitive transducer is R, the acoustic lens is formed with a material and a thickness that satisfy 10 Log₁₀(R+1)/f_{c}<αd. As mentioned above, the FBW of the sole capacitive transducer is nearly 100%. The upper-limit frequency of a bandwidth is set to 1.5fc with the fractional bandwidth regarded as 100%. Under FBW=100% and δ=0.5, L<1/((3πf_{c})²×C) of the inequality (12) can be rewritten into an equation (13) below.

Specifically, assuming that an inductance value per channel of a capacitive transducer is L [H], an electrostatic capacity per channel of the capacitive transducer is C [pF], the center frequency of the capacitive transducer is f_{c} [MHz], FBW denotes a fractional bandwidth, and δ denotes a coefficient determining an LC resonant frequency ω_{LC} concerning a stray capacitance C′ and the series inductor 21, the series inductor is constructed to exhibit an inductance value satisfying the equation (13) below.

L=1/((2π(1+δFBW)f_{c})²×C′) (13)

The stray capacitance C′ is a capacitance including a capacitance C of a device effective as a sensor for the capacitor 31 and a parasitic capacitance due to wiring.

δ takes on a value ranging from 0.3 to 1.0. Especially, under δ≧0.5, a pulse-characteristic remedial effect is great. Under δ<0.5, the resonant frequency falls within a sensitivity band, the phase linearity is lost, and the pulse characteristic is gradually deteriorated. In contrast, when δ is too large, a resonant condition is met at a frequency far away from the sensitivity band. Therefore, the sensitivity remedial effect due to an inductance is gradually lost.

### BRIEF DESCRIPTION OF THE DRAWINGS

FIG. 1 It is a configuration diagram showing an embodiment of an ultrasound probe in accordance with the present invention.

FIG. 2 It is a diagram of an electrical equivalent circuit for reception equivalent to one channel of a capacitive transducer.

FIG. 3 It is a table listing constants for the electrical equivalent circuit of the capacitive transducer.

FIG. 4 It is a characteristic diagram showing a scattering parameter S in relation to a frequency.

FIG. 5 It is an outline diagram concerning reduction of multiple echoes by an acoustic lens.

FIG. 6 It includes pulse characteristic diagrams and frequency characteristic diagrams concerning an ultrasound probe unrelated to the present invention.

FIG. 7 It includes pulse characteristic diagrams and frequency characteristic diagrams concerning an ultrasound probe in accordance with the present invention.

FIG. 8 It is a comparison table for a transmit/receive gain and a multiple-echo level.

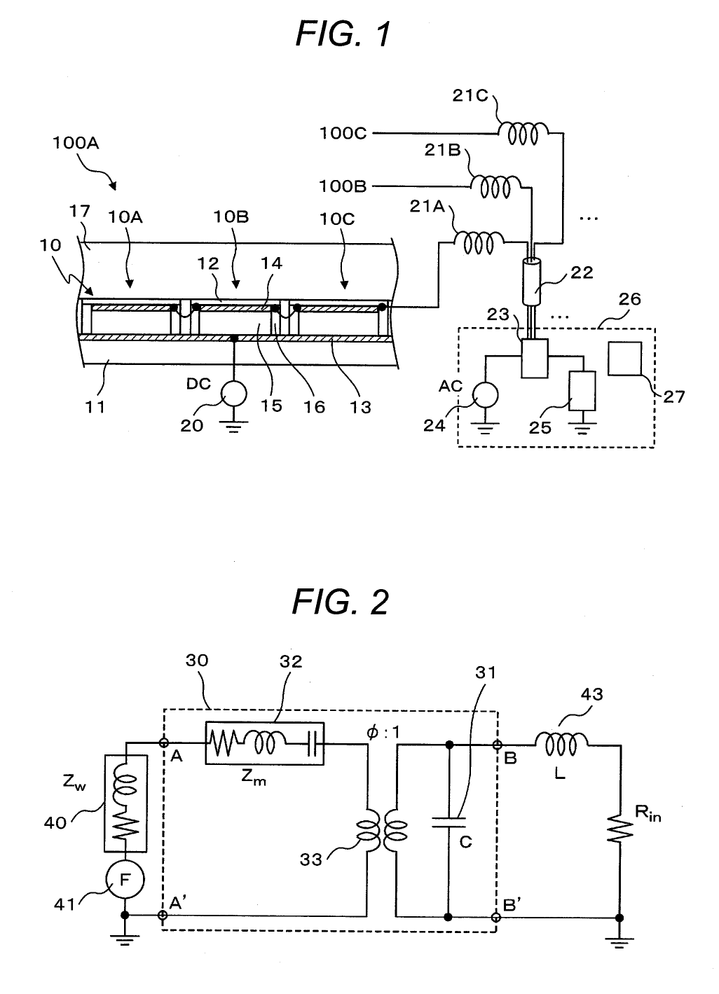

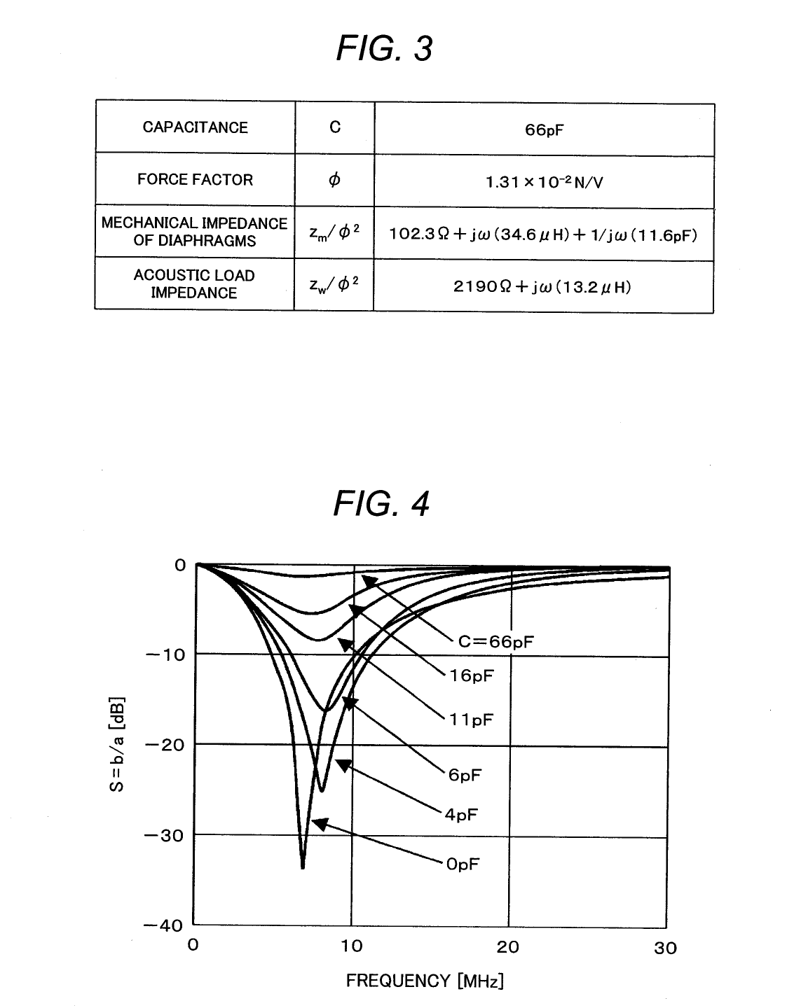

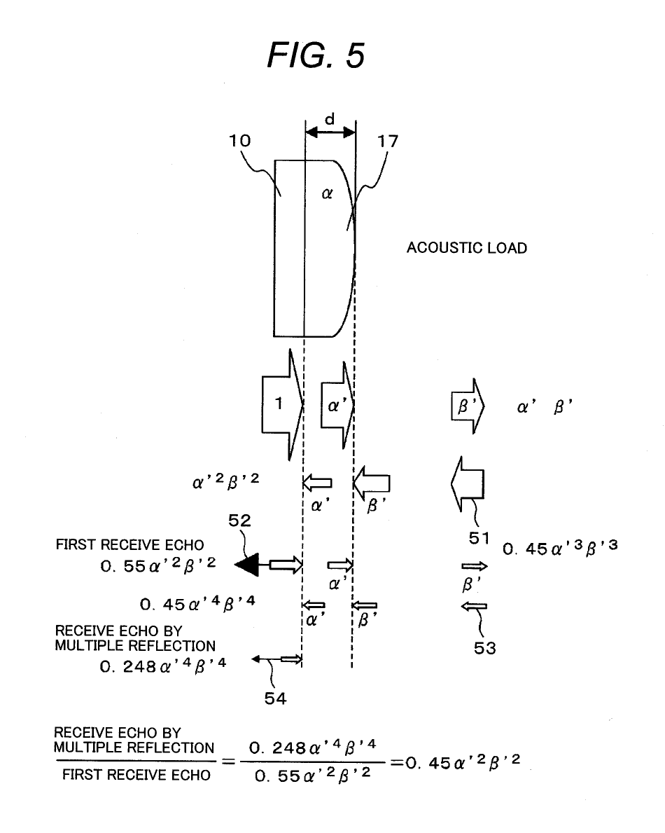

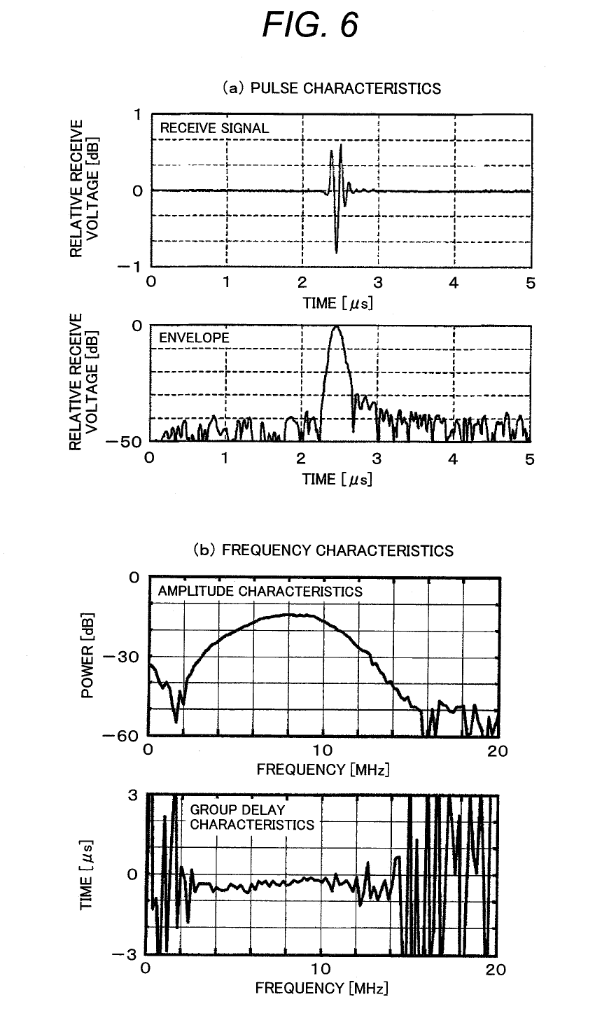

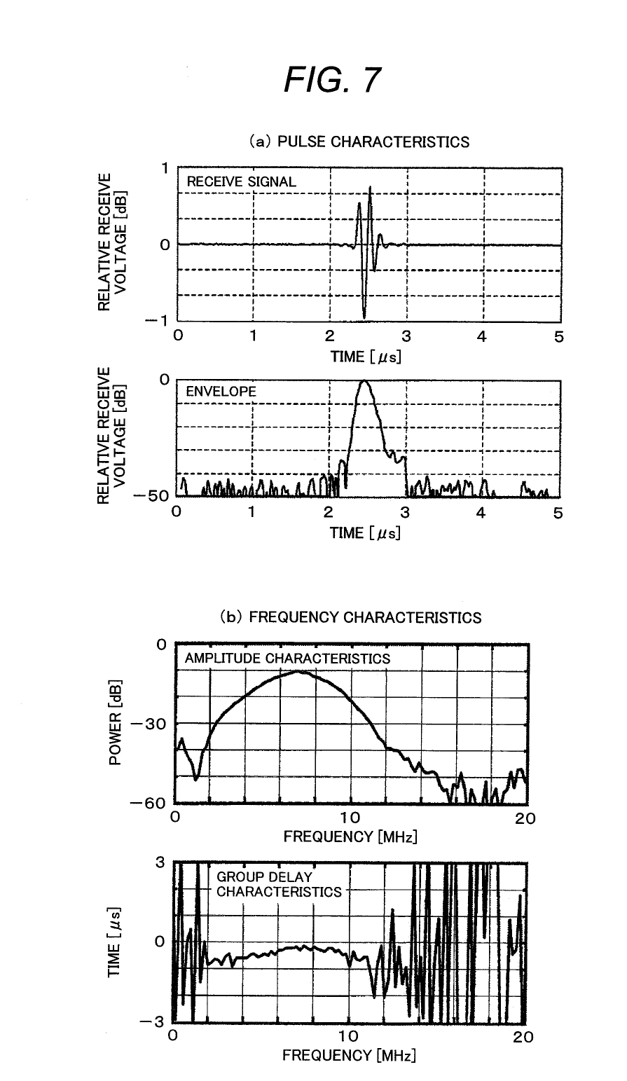

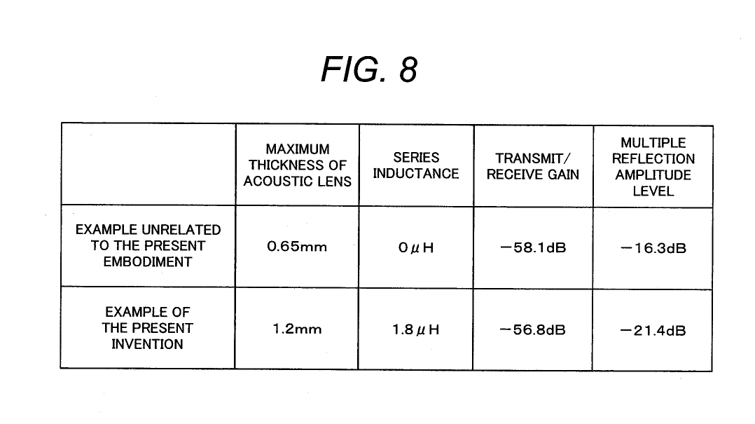

### EXPLANATION OF REFERENCE NUMERALS

10 . . . capacitive transducer
10A, 10B, 10C . . . diaphragm structure
11 . . . silicon substrate
12 . . . diaphragm
13 . . . lower electrode
14 . . . upper electrode
15 . . . layer of cavity
16 . . . rib
17 . . . acoustic lens
20 . . . DC electrical source
21 . . . series inductor
22 . . . probe cable
23 . . . transmitter/receiver circuit
24 . . . AC electrical source
25 . . . receiver circuit
30 . . . part of electrical equivalent circuit of capacitive transducer
31 . . . capacitor
32 . . . mechanical impedance of diaphragms
40 . . . acoustic load impedance
41 . . . driving force by arriving sound
42 . . . receive impedance
51 . . . first reflection echo
52 . . . first receive echo
53 . . . multiple reflection echo
54 . . . receive echo by multiple reflection

1. An ultrasound probe comprising:
a capacitive transducer including at least one channel formed on a substrate;
an acoustic lens disposed on one surface of the capacitive transducer; and
a series inductor connected in series with each channel of the capacitive transducer,
wherein the acoustic lens is formed with the absorption coefficient of the acoustic lens and the maximum thickness of the acoustic lens based on the resonant frequency of the capacitive transducer; and
wherein, as for the inductance value of the series inductor, the inductance value per channel of the capacitive transducer is specified based on the electrostatic capacity per channel of the capacitive transducer and the center frequency of the capacitive transducer.

2. The ultrasound probe according to claim 1,
wherein, assuming that the absorption coefficient of the acoustic lens is α [dB/mm/MHz], the maximum thickness of the acoustic lens is d [mm], and the resonant frequency of the capacitive transducer is f_{c} [MHz], the acoustic lens satisfies the relationship of 6.5/f_{c}<αd.

3. The ultrasound probe according to claim 1,
wherein, assuming that the inductance value per channel of the capacitive transducer is L [H], the electrostatic capacity per channel of the capacitive transducer is C [pF], and the center frequency of the capacitive transducer is f_{c} [MHz], the inductance value of the series inductor satisfies the relationship of L<1/((3πf_{c})²×C).

4. The ultrasound probe according to claim 1, comprising a plurality of capacitive transducer array channels formed on the substrate,
wherein each series inductor is connected in series with the respective capacitive transducer array channel.

5. The ultrasound probe according to claim 1, further comprising a cable over which an ultrasound signal is drawn out,
wherein the series inductor has one terminal thereof connected to the channel of the capacitive transducer, and has the other terminal thereof connected onto the cable.

6. The ultrasound probe according to claim 1, wherein the material of the acoustic lens is silicon rubber.

7. The ultrasound probe according to claim 1, wherein the absorption coefficient of the acoustic lens is equal to or larger than 0.7 dB/mm/MHz and equal to or smaller than about 1 dB/mm/MHz.

8. The ultrasound probe according to claim 1, wherein the maximum thickness of the acoustic lens is equal to or larger than 0.73 mm.

9. An ultrasound imaging apparatus comprising:
a capacitive transducer including a plurality of channels formed on a substrate;
an ultrasound probe including an acoustic lens disposed on one surface of the capacitive transducer and an inductor connected to the capacitive transducer;
an AC electrical source that applies an AC voltage to the ultrasound probe; and
a receiver circuit that processes a receiving signal of the ultrasound probe,
wherein the ultrasound probe is the ultrasound probe set forth in claim 1.

10. The ultrasound imaging apparatus according to claim 9, further comprising a transmitter/receiver circuit and a controller,
wherein, for transmitting ultrasound waves, the controller connects the transmitter/receiver circuit to the AC electrical source and inductor; and
wherein, for receiving ultrasound waves, the controller connects the transmitter/receiver circuit to the receiver circuit and conductor.
